(19) United States
(12) Patent Application Publication
(10) Pub. No.: US 2025/0066737 A1
(43) Pub. Date: Feb. 27, 2025

(54) METHOD FOR ESTABLISHING LUNG CANCER CELL MODEL HAVING VASCULAR TUBES, PLATFORM FOR LUNG CANCER MEDICINE SCREENING AND METHOD FOR USING THEREOF

(71) Applicant: National Cheng Kung University Hospital, Tainan (TW)

(72) Inventor: Chun-Hui Lee, Tainan (TW)

(21) Appl. No.: 18/812,000

(22) Filed: Aug. 22, 2024

Related U.S. Application Data
(60) Provisional application No. 63/534,576, filed on Aug. 24, 2023.

(51) Int. Cl. C12N 5/09; G01N 33/50

(52) U.S. Cl. CPC C12N 5/0693; G01N 33/5011; C12N 2500/02; C12N 2503/02; C12N 2513/00

(57) ABSTRACT

The present invention provides a method for establishing lung cancer cell model having vascular tubes, wherein the lung cancer cell model can be subjected to live cell real-time dynamic image analysis or fixed cell static image analysis. The method comprises: seeding a cell suspension between an upper 3D culture substrate and a lower 3D culture substrate for cultivation into a lung cancer cell model, wherein the upper 3D culture substrate and the lower 3D culture substrate are both growth factor-reduced culture media and volume of the upper 3D culture substrate is larger than volume of the lower 3D culture substrate. The lung cancer model prepared by the aforesaid method can be further applied to patency validation of a 3D-VM by using fluorophore-tagged microsphere, and to establishment of an anti-VM drug screening platform and a screening method therefor.

### FIELD OF THE INVENTION

The present invention relates to three-dimensional cell culture technology, particularly to a three-dimensional cell culture method for simulating tumor formation by using cancer cells, and further relates to imaging method for validation of vascularization and tube formation of cancer cells; in another aspect, the present invention involves a use of the tumor-simulating cell model for establishment of a platform for medicine screening and a method for using thereof.

### BACKGROUND OF THE INVENTION

The aggressiveness of malignancies is determined by the blood supply, which enables their growth and facilitates hematogenous spread. Angiogenesis, a process extensively studied over the years, was first proposed as a theory by Folkman et al. in 1971, marking a significant advancement in cancer treatment strategies1. Despite this progress, clinical trials with anti-angiogenic agents, such as bevacizumab (a VEGF-targeting antibody), have often yielded disappointing results, with most patients experiencing only transient responses followed by resistance.

Vasculogenic mimicry (VM), first described in uveal melanoma, refers to the ability of aggressive tumor cells to transdifferentiate and adopt endothelial cell characteristics, forming novel vascular networks and an autonomous microcirculation independent of nonmalignant host cells. VM has been observed in various cancers and is generally associated with poor prognosis. Several characteristics of tumor cells have been postulated as causes of VM formation—the plasticity of tumor cells and hypoxic tumor microenvironment (TME). The acquired properties may include epithelial-to-mesenchymal transition (EMT) and cancer stemness, which might function together to enhance the capacity for tubule formation. The main signaling pathways associated with VM are the VE-cadherin (VE-Cad), Notch, and Hypoxia-inducible factor (HIF) pathways, which promote VM formation and invasiveness.

Lung cancer is the leading cause of cancer mortality worldwide. Non-small cell lung cancer (NSCLC), which accounts for around 85% of all lung cancer, often has a dismal prognosis with a 5-year survival rate of less than 15%. In Taiwan, epidermal growth factor receptor (EGFR) mutation emerges as the most common driver (approximately 50%) in lung adenocarcinoma (LUAD). This subpopulation of individuals survives longer with EGFR tyrosine kinase inhibitors (TKIs). Treatment resistance is a crucial obstacle to cancer management. However, limited biochemical and molecular details are known concerning lung cancer tumor cells' capacity to generate intricate vascular channels. Recent studies have unveiled that VM can serve as an alternative blood supply when angiogenesis is inhibited, rendering VM a promising new aspect for targeting tumors.

Foslinanib, a novel small-molecule anti-vasculogenic mimicry (VM) compound, exhibited robust anti-tumor efficacy in various tumor xenograft models by inducing cell cycle arrest and apoptosis and suppressing the formation of VM tubules in tumor cells.

Since its first report by Maniotis in 1999, the existence of VM has been a subject of considerable debate. Two distinct types of VM—“tubular type” and “patterned matrix type”—have been reported in the literature, shedding light on their characteristics and implications4. Although the presence of VM in vivo, in both animal models and patient tumors, is often inferred from intense positive staining for glycoproteins using Periodic Acid-Schiff (PAS) stain, this interpretation is not universally accepted. The contentious debate extends to the existence of an in vitro model of VM, which has sharply divided the scientific community. Initial reports indicated that channels or tubes form in cancer cell monolayers cultured on Matrigel, and these structures might support fluid movement. Subsequent studies proposed that intercellular connections in cancer cells grown on Matrigel represented VM. This interpretation appears to have gained traction within the cancer research community, leading to a preponderance of scientific literature that reports both the presence and mechanisms of VM based on these intercellular connections rather than the presence of fluid-conducting tubes.

### SUMMARY OF THE INVENTION

As validation of tube formation and tube patency in VM vasculature draws less attention from the mainstream science research community, the effectiveness of anti-cancer medicine screening using the cell models having VM vasculature without validation remains up for debate.

Accordingly, in the present invention, a breakthrough medicine testing platform is developed for evaluating VM behavior and exploring new types of anti-VM medicine, especially those targeting lung cancer. A cell model having complex 3D-VM vasculature is established using three-dimensional scaffold prepared by using materials mimicking basement membrane components. The cell model allows real-time dynamic imaging analysis of live cells or static imaging analysis of fixed cells. In addition to revealing the complex tube structure formed of dynamic tumor cells, the present invention further provides a method for validating the cell model having functional 3D-VM vasculature by using high-throughput microscopy, and the cell model is further applied to anti-VM medicine screening.

In one aspect, the present invention discloses a method for establishing a lung cancer cell model having vascular tubules, comprising: seeding a cell suspension between an upper 3D culture substrate and a lower 3D culture substrate to obtain a pre-cell model, wherein the cell suspension comprises lung cancer cells; and incubate the pre-cell model for a cultivation time so as to obtain the lung cancer cell model, wherein: both the upper 3D culture substrate and the lower 3D culture substrate are growth factor-reduced culture substrates, and volume of the upper 3D culture substrate is larger than volume of the lower 3D culture substrate.

In some embodiments, the lung cancer cell comprises first lung cancer cells expressing wild-type EGFR at a first basal level or the first lung cancer cells carrying a first exogenous genetic material to express wild-type EGFR at a first expression level, wherein the first expression level is larger than the first basal level; and second lung cancer cells expressing mutant EGFR at a second basal level, or the second lung cancer cells carrying a second exogenous genetic material to express mutant EGFR at a second expression level, wherein the second expression level is larger than the second basal level.

In some embodiments, the cell suspension further comprises microspheres labeled with first fluorophores, and each of the microspheres comprises an electronegative organic group; the lung cancer cells are further labeled with second fluorophores having emission light different from the emission light of the first fluorophores; and the vascular tubule comprises a lumen surrounded by the lung cancer cells and a tunica adventitia formed by the lung cancer cells surrounding on the lumen, wherein the method further comprises:

In another aspect, the present invention discloses a platform for lung cancer medicine screening comprising a lung cancer cell model prepared by the aforesaid method.

In yet another aspect, the present invention discloses a method for screening lung cancer medicines, comprising: providing a lung cancer cell model to be tested having vasculature of a first vascular density, wherein the lung cancer cell model to be tested is prepared by the aforesaid method; co-culturing the lung cancer cell model to be tested and an anti-cancer medicine for a screening time so as to obtain a tested lung cancer cell model having vasculature of a second vascular density; and evaluating quantitative relationship between the first vascular density and the second vascular density to determine whether the anti-cancer medicine is therapeutically effective, wherein: when a ratio of the first vascular density to the second vascular density is larger than 1, determining the anti-cancer medicine to be therapeutically effective; when a ratio of the first vascular density to the second vascular density is equal to or smaller than 1, determining the anti-cancer medicine to be therapeutically ineffective.

### BRIEF DESCRIPTION OF THE DRAWINGS

FIG. 1A is a cross-sectional diagram illustrating the stratification of a lung cancer cell model;

FIG. 1B is a flowchart illustrating a method for establishing a lung cancer cell model in the first aspect of the present invention;

FIG. 1C to 1F presents a flowchart and three-dimensional diagrams to elaborate a method for establishing a pre-cell model;

FIG. 2A is a flowchart to illustrate a method for establishing a lung cancer cell model in the second aspect of the present invention;

FIG. 2B is a cross-sectional diagram illustrating the structure of a vasculature;

FIG. 2C is a cartoon illustrating a workflow of three-dimensional imaging of the vasculature under fluorescence microscopy.

FIG. 2D is a flowchart illustrating the steps of the vasculature patency evaluating process;

FIG. 2E is a cross-sectional diagram illustrating standards for determination of vasculature with valid or invalid patency;

FIG. 3 is a flowchart illustrating a method for screening lung cancer medicines;

FIG. 4 demonstrates cell morphology of a stable clone before and after FACS screening under fluorescence microscopy;

FIG. 5A is a cartoon illustrating the observation perspective of VM network and lumens in Example 1;

FIG. 5B presents bright field images illustrating VM network formation by a lung cancer cell line, and the images were captured before and after PAS staining;

FIG. 5C presents overlay of fluorescence microscopy images demonstrating vasculature within a cell model established with the stable clone;

FIG. 5D presents live cell fluorescence microscopy images demonstrating comparative analysis of vasculature topological features within cell models of non-small cell lung cancer cell lines transfected by EGF;

FIG. 6 presents an overlay of live cell fluorescence microscopy images demonstrating vasculatures formed of H1299 cells and A549 cells in normoxia or hypoxia;

FIG. 7A presents an overlay of live cell fluorescence microscopy images demonstrating the localization of carboxylate-modified microspheres and amine-modified microspheres within a 3D-VM vasculature, respectively;

FIGS. 7B to 7C present fluorescence microscopy images demonstrating the carboxylate-modified microspheres in x-z cross-section and y-z cross-section of a VM tube within HCC827 lung cancer cell models, respectively, FIG. 8A presents live cell fluorescence microscopy images demonstrating changes of 3D-VM vasculature within H1650 and HCC827 lung cancer cell models under various foslinanib concentrations;

FIG. 8B presents bar charts and box plots illustrating quantity analysis of changes in cell viability and 3D-VM vasculature in H1650 and HCC827 lung cancer cell models under various foslinanib concentrations;

FIG. 9A is a box plot illustrating the difference in VM ssGSEA scores between NSCLC and SCLC;

FIG. 9B is a box plot to illustrate the difference in VM ssGSEA scores between LUAD cases of wild-type EGFR and mutant EGFR;

FIG. 9C presents an overlay of live cell fluorescence microscopy images and cross-sectional diagrams demonstrating VM vasculature within H1299-EGFRwt and H1299-EGFRmt cell models, respectively;

FIG. 9D presents a box plot illustrating the quantitative analysis of functional lumen structures in 3D-VM vasculature within H1299-EGFRwt and H1299-EGFRmt cell models;

FIG. 10A presents fluorescence microscopy images and bar charts illustrating changes in cell viability and 2D-VM networks within H1299-EGFRmt, H1299-EGFRwt, and H1299 cell models post foslinanib treatment;

FIG. 10B presents 3D overlays of fluorescence microscopy images demonstrating changes in 3D-VM vasculatures within H1299-EGFRmt, H1299-EGFRwt, and H1299 cell models upon single use of foslinanib, osimertinib, and a combined use thereof, respectively.

FIG. 10C is a bar chart presenting quantitative analysis illustrating changes in 3D-VM vasculature upon single use of foslinanib, osimertinib, and a combined use thereof, respectively.

FIG. 11A presents immunohistochemical staining diagrams illustrating vasculature formation and LAMC2 expression within EGFRmt human tumor tissue; and

FIG. 11B presents Western blotting and bar charts illustrating quantitative analysis of changes in total expression levels of EGFR, LAMC2 and phosphorylation of EGFR within H1299 cell model upon single use of foslinanib or osimertinib, and a combined use thereof.

### DETAILED DESCRIPTION OF THE INVENTION

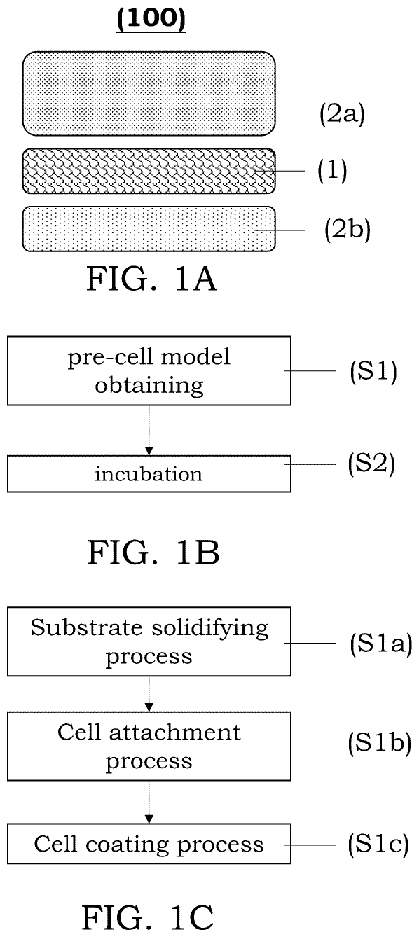

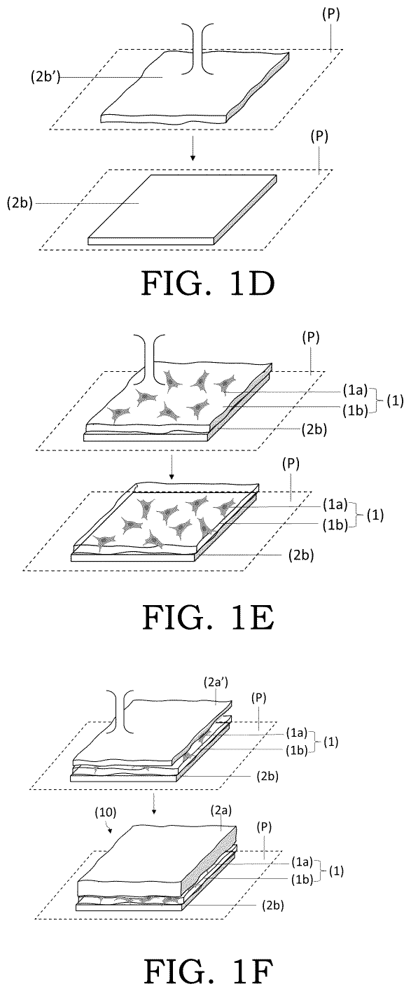

The first aspect of the present invention is to provide a method for establishing a lung cancer cell model (100) having vascular tubules, wherein the lung cancer cell model (100) serves as a platform for dynamic imaging analysis of live cells or static imaging analysis of fixed cells. Referring to FIGS. 1A to 1B, the method comprises: pre-cell model obtaining step (S1): seeding a cell suspension (1) between an upper 3D culture substrate (2a) and a lower 3D culture substrate (2b) to obtain a pre-cell model (10), wherein the cell suspension (1) comprises lung cancer cells (la); and incubation step (S2): incubating the pre-cell model (10) for a cultivation time to obtain the lung cancer cell model (100), wherein: both the upper 3D culture substrate (2a) and the lower 3D culture substrate (2b) are growth factor growth factor-reduced culture substrates, and volume of the upper 3D culture substrate (2a) is larger than the volume of the lower 3D culture substrate (2b).

In various embodiments, to allow the lung cancer cells to migrate, invade, and differentiate into three-dimensional vasculature, conceivably, the upper 3D culture substrate (2a) and the lower 3D culture substrate (2b) are tissue scaffolds with porosity. To simulate cancer cells migrating, invading and differentiating in vivo, specifically the three-dimensional topography of extracellular matrix, the upper 3D culture substrate (2a) and the lower 3D culture substrate (2b) can be porous layer in a gel form. Depending on tissue types, the exemplified extracellular matrix can be an interstitial matrix, pericellular matrix, cartilage matrix, bone matrix, tendon and ligament matrix, dental Matrix, or basement membrane.

In certain embodiments, as shown in FIGS. 1C to 1F, the pre-cell model (10) obtaining step (S1) further comprises:
substrate solidifying process (Sla): solidifying a lower volume of a lower 3D culture pre-substrate (2b′) on a culture surface (P) for a solidifying time so that the lower 3D culture substrate (2b) is formed;
cell attachment process (S1b): incubating a middle volume of the cell suspension (1) on the lower 3D culture substrate (2b) for an attachment time so that the lung cancer cells (la) are attached to the lower 3D culture substrate (2b); and
cell coating process (S1c): coating an upper volume of an upper 3D culture pre-substrate (2a′) onto the lung cancer cells (la) so that the upper 3D culture substrate (2a) is formed, thereby obtaining the pre-cell model (10), wherein the upper volume is larger than the sum of the lower volume and the middle volume.

Please continue with reference to FIGS. 1E to 1F, the cell suspension (1) is a mixture made by thoroughly dispersing the lung cancer cells (la) and a suspending solution (1b). The suspending solution (1b) can be an isotonic physiological buffer applicable for cell cultures, such as DPBS, EBSS, HBSS, HEPES, or PBS. To maintain the viability of the lung cancer cells (la), the suspending solution (1b) can be a cell culture medium supplemented with essential nutrients, such as DMEM, RPMI-1648, F12K, or EMEM. To prevent the lung cancer cells (la) from degeneration or differentiation, other essential nutrients, including fetal bovine serum or growth factors, can also be supplemented to the suspending solution (1b). In various embodiments, the suspending solution (1b) is cell culture medium, preferably RPMI-1640, required by the lung cancer cells (la).

In various embodiments, to simulate the lung cancer cells' gradual differentiation into cells with epithelial and endothelial characteristics during three-dimensional culture, which stimulates angiogenesis and vascular mimicry formation so that observable vasculature is developed within the cultivation time. Specifically, the upper 3D culture pre-substrate (2a′) and the lower 3D culture pre-substrate (2b′) are used for mimicking basement membrane.

In preferred embodiments, the upper 3D culture pre-substrate (2a′) is prepared by mixing a first upper volume of upper cell basement membrane-like materials and a second upper volume of a cell culture medium, wherein the first upper volume is the same as or smaller than the second upper volume and the sum of the first upper volume and the second upper volume is equal to the upper volume. The lower 3D culture pre-substrate (2b′) comprises lower cell basement membrane-like materials, and the upper cell basement membrane-like materials are the same as or different from the lower cell basement membrane-like materials. Preferably, the upper cell basement membrane-like materials are the same as the lower cell basement membrane-like materials.

Specifically speaking, based on the fact that the volume of the upper 3D culture pre-substrate (2a′) is larger than the volume of the lower 3D culture pre-substrate (2b′), the volume of the upper 3D culture pre-substrate (2a′) provides larger space of migration and growth for the lung cancer cells (la). In addition, since the upper 3D culture pre-substrate (2a′) and the cell culture medium can be mixed in equal volumes (1:1) or mixed with the cell culture medium in a smaller volume, such as 1:(1.1 to 3), the concentration of the upper cell basement membrane-like materials is lower than the concentration of the lower cell basement membrane-like materials in the lower 3D culture pre-substrate (2b′). As such, during the subsequent cultivation, the upper cell basement membrane-like materials form a three-dimensional structure mimicking the basement membrane with a looser structure.

When in vivo metastasis of cancer cells occurs, ECM remodeling also occurs to create an environment beneficial for the growth and differentiation of the cancer cells, involving degradation and renewal of the extracellular matrix. Accordingly, based on the concentration gradient of the basement membrane-like materials across the upper 3D culture pre-substrate (2a′) and the lower 3D culture pre-substrate (2b′), a microenvironment for oriented ECM remodeling can be simulated. The concentration of the basement membrane-like materials decreases from the lower 3D culture pre-substrate (2b′) to the upper 3D culture pre-substrate (2a′). As such, the lung cancer cells (la) are induced to migrate towards the upper 3D culture pre-substrate (2a′) due to its loose structure and differentiate into epithelium-like cells or endothelium-like cells so as to form the vasculature (3).

In preferred embodiments, the upper basement membrane-like materials are components of a basement membrane, such as fibronectin, collagen, laminin, vitronectin, polylysine, proteoglycan sulfate, nestin, or a combination of two or more thereof. Similarly, the lower basement membrane-like materials are components of a basement membrane, such as fibronectin, collagen, laminin, vitronectin, polylysine, proteoglycan sulfate, nestin, or a combination of two or more thereof. In one preferred example, Matrigel, rich in laminin and collagen IV, is applied as the upper 3D culture pre-substrate (2a′) and the lower 3D culture pre-substrate (2b′).

In some embodiments, a ratio of the first upper volume, the second upper volume, the middle volume, and the lower volume is 1:(1.00 to 3.00):(0.5 to 2.00):(0.3 to 1.00). Preferably, the ratio is 1:(1.00 to 1.50):(0.8 to 1.5):(0.4 to 0.8). More preferably, the ratio is 1:1:1:(0.6 to 0.7).

In some embodiments, the coating density of the upper 3D culture pre-substrate (2a′) is 130 to 350 μL/cm², and preferably is 135 to 180 μL/cm². A coating density of the lower 3D culture pre-substrate (2b′) is 50 to 150 μL/cm², and preferably 80 to 120 μL/cm². The ratio of the coating density of the upper 3D culture pre-substrate (2a′) to the coating density of the lower 3D culture pre-substrate (2b′) is (0.80 to 2.60): 1, and preferably is (1.00 to 1.50): 1. The lung cancer cells are seeded onto the lower 3D culture substrate (2b) at a cell density of 4.0×10⁴ to 7.0×10⁴ cells/cm², and preferably of 5.0×10⁴ to 6.0×10⁴ cells/cm².

In one example, Trulive3D dish (Bruker) is applied, and each culture well thereof is added with 50 to 150 μL the lower 3D culture pre-substrate (2b′) so as to form the lower 3D culture substrate (2b) after solidification. Preferably, 100 μL or 150 μL of the lower 3D culture pre-substrate (2b′) is added. Subsequently, 100 to 300 μL of the cell suspension (1) is seeded in each of the wells to allow attachment of the lung cancer cells. Preferably, 100 μL, 150 μL, 200 μL or 250 μL of the cell suspension (1) is seeded. The upper 3D culture pre-substrate (2a′) is prepared by mixing 150 to 300 μL of the cell culture medium with 150 μL to 450 μL of the upper basement membrane-like materials. Preferably, 150 μL, 200 μL, 250 μL or 300 μL of the cell culture medium are mixed with 150 μL, 200 μL, 250 μL, 300 μL, 350 μL, 400 μL or 450 μL of the upper basement membrane-like materials. Subsequently, the upper 3D culture pre-substrate (2a′) is coated onto the attached lung cancer cells (la) so that the pre-cell model (10) is accomplished.

In various embodiments, the lung cancer cells (la) are derived from lung cancer tissue or lung cancer cell lines, wherein the lung cancer tissue can be retrieved from cryo-preserved or freshly removed lung cancer tumors. Lung cancer includes small-cell lung cancer, large-cell lung cancer, squamous-cell lung carcinoma. or lung adenocarcinoma. On the other hand, the lung cancer cell line includes immortalized cell lines derived from any one of the aforesaid lung cancers, and is not limited to this. In some preferred embodiments, the lung cancer cell line can be A549, HCC827, H460, H1299, H1650 or H1975.

In various embodiments, the cultivation time varies with lung cancer cell types. Specifically, the cultivation time is 4 to 72 hours, and preferably 4 to 6 hours, 6 to 12 hours, 12 to 24 hours, 24 to 36 hours, 36 to 48 hours, 48 to 60 hours or 60 to 72 hours, and more preferably 4 to 6 hours, 12 to 24 hours or 36 to 48 hours. The solidification time varies with types of the lower basement membrane-like materials. Specifically, the solidification time is 5 to 60 minutes, preferably 10 to 40 minutes, and more preferably 15 to 30 minutes. The attachment time varies with lung cancer cell types. Specifically, the attachment time is 5 to 60 minutes, preferably 10 to 40 minutes, and more preferably 15 to 30 minutes, wherein the attachment time is the same as the solidification time.

In various embodiments, the incubation step (S2), the substrate solidifying process (Sla), the cell attachment process (S1b) can be conducted at room temperature or physiological temperature, and particularly at 25 to 37° C. . . . In another aspect, to create an environment simulating cell physiological conditions and thereby maintaining the buffering ability of the cell culture medium, comprehensively, the incubation step (S2) and the cell attachment process (S1b) are both conducted in an atmosphere of 5% CO₂.

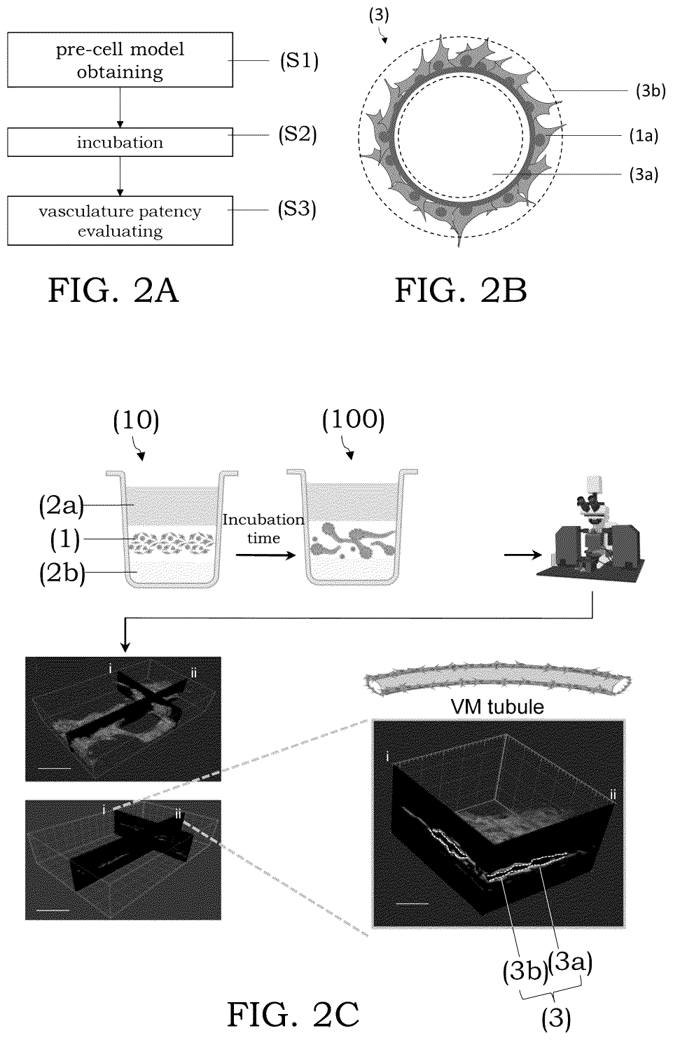

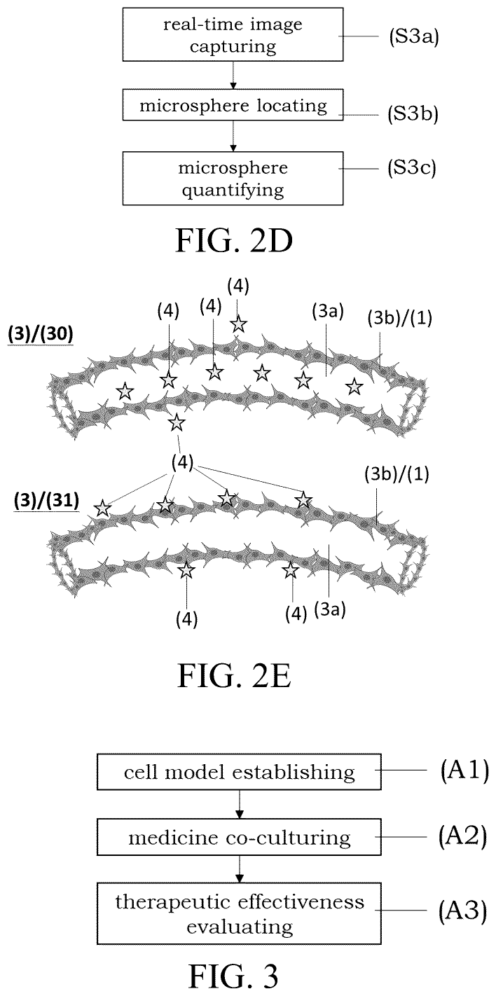

With reference to FIG. 2A, the second aspect of the present invention is to provide a method for establishing a lung cancer cell model (100) having vascular tubules, and the steps, cell types, materials, culturing conditions, and other parameters thereof are overall the same as those in the first aspect of the present invention, but the method further comprises a vasculature patency evaluating process (S3). Tubular functions of vascular tubes in the lung cancer cell model (100) are further evaluated, particularly the tubular structure being functional in assisting lung cancer cells with nutrient transportation, exchange, and removal of metabolites, wherein the vasculature specifically refers to vascular mimicry.

In various embodiments, the vasculature patency evaluating process (S3) is conducted based on live cell imaging, particularly using fluorescent microscopy to observe and evaluate the morphology of cells and tubular structures in real time. As shown in FIG. 2B, the vasculature (3) comprises a lumen surrounded by the lung cancer cells (la) and an outer tunica (3b) formed of the lung cancer cells (la) by surrounding the lumen (3a). To distinguish the lung cancer cell (la) forming the outer tunica (3b) and the lumen (3a), according to the high-contrast principle, the lung cancer cell (la) is labeled with first fluorophores such as GFP, RFP, YFP or other types of fluorophores, and not limited to this.

Notably, the vasculature (3) is a 3D structure, and a 3D imaging of the vasculature (3) is the focus of analysis in the present invention. In one exemplary embodiment, as shown in FIG. 2C, the 3D imaging of the vasculature (3) can be obtained from x-z cross-section (i plane) and y-z cross-section (ii plane), wherein the lumen (3a) is a cell-free lumen structure outlined by white dot line, and the outer tunica (3b), formed of the lung cancer cells (la), is a fluorophore-labeled area surrounding the lumen (3a).

In some embodiments, as for real-time observation of biological fluid circulating in the functional lumen (3a), the biological fluid within the lumen (3a) is further required to be detectable under fluorescent microscopy. Therefore, in order to label the biological fluid, microspheres (4) are added to the cell suspension (2) in the cell attachment process (S1b), wherein the microspheres (4) are labeled with second fluorophores having emission light different from the emission light of the first fluorophores. The second fluorophores can be GFP, RFP, YFP, or other types of fluorophores, and not limited to this.

In various embodiments, with reference to FIGS. 2D and 2E, the vasculature patency evaluating process (S3) comprises:
real-time image capturing step (3a): capturing a real-time image of the vasculature (3) in the lung cancer cell model (100), wherein, in the real-time image, the microspheres (4) is imaged with a first color corresponding to the first fluorophores, and the vasculature (3) is imaged with a second color corresponding to the second fluorophores; and
microsphere locating step (S3b): locating the microspheres (4) in the real-time image according to the relative position of the first color and the second color;
microsphere quantifying step (S3c): quantifying the microspheres (4) localized in the vasculature (3) so as to evaluate the patency of the vasculature (3), wherein a first quantity of the microspheres (4) localized in the lumen (3a) is quantified, and a second quantity of the microspheres (4) upon the outer tunica (3b) or on the outer side of the outer tunica (3b) is quantified, wherein: when the first quantity is larger or the same as the second quantity, determining the vasculature (3) to be a vasculature with valid patency (30); when the first quantity is smaller than the second quantity, determining the vasculature (3) to be a vasculature with invalid patency (31).

As a matter of course, during the formation of vasculature (3), migration and differentiation of the lung cancer cells (la) changes the relative position between the lung cancer cells (la) and the microspheres (4). At the mean time, when the lung cancer cells (la) form the outer tunica (3b), the lumen (3a), as defined by the surrounding outer tunica (3b), would engulf the microspheres (4) into the inner side. As biological fluid begins to flow inside the lumen (3a), a displacement of the microspheres (4) along the flow direction of the biological fluid can be observed in real-time imaging inside the lumen (3a), but the microspheres (4) would not cross the boundary defined by the outer tunica (3b). Moreover, at the incubation step (S2), in addition to the microspheres (4) engulfed by the lumen (3a) owing to formation of the outer tunica (3b), based on the microspheres' (4) hydrophilic, hydrophobic, or electrical properties, a portion of the microspheres (4) on the outer side of the outer tunica (3b) would enter the lumen (3a) across the outer tunica (3b) since the lung cancer cells (la) can bridge exchanges of biological substances inside the lumen (3a) and biological substances on the outer side of the outer tunica (3b). Therefore, the quantity of the microspheres (4) inside the lumen (3a) is supposed to be larger than the quantity of the microspheres (4) on the outer side of the outer tunica. Nonetheless, if there is a large number of microspheres (4) attaching to the outer tunica (3b), it indicates that a vascular structure similar to VM is formed in the lung cancer cell model (100), but the exchange of biological substances across the outer tunica (3a) is inefficient or never occurs, or the vacant spaces similar to the lumen are simply intercellular spaces. Such a vascular structure, without tubules of patency, is unfunctional.

In some embodiments, to enable the microspheres (4) to cross the cell membrane and join the cross-outer tunica (3) exchange of biological substances, the microspheres (4) further comprise an organic group having negativity, wherein the organic group having negativity can be exemplified by a carboxyl group (—COO—), a nitro group (—NO₂), a sulfoxide group (—SO—), a cyano group (—CN—) or a sulfo group (—SO₂), preferably the carboxyl group (—COO—).

In general, vasculature formed in VM can be categorized by microvascular channel or macrovascular channel. The tube diameters are 5 to 10 μm and 15 to 30 μm, respectively. VM formed during cancer cell migration is a dynamic vasculature, and the tube diameters changes with the tumor progressions, but remains within the aforementioned diameter range. Misjudgment may occur when sizes of the microspheres (4) are too large to enter the lumens (3a), or when the sizes are so small that the microspheres merely enter the intercellular spaces. As such, to ensure entry of the microspheres (4) into the lumen (3a) and to raise its confidence, diameters of the microspheres (4) are 0.3 to 2.0 μm, and preferably 0.5 to 1.0 μm.

The third aspect of the present invention is to provide a method for establishing a lung cancer cell model (100) having vascular tubules, and the steps, cell types, materials, culturing conditions and other parameters thereof are overall the same as those in the first aspect or the second aspect of the present invention, but the pre-cell model (10) is incubated in normoxia, physioxia or hypoxia, wherein atmospheric condition of normoxia is 5% CO₂ and 18 to 22% O₂, atmospheric condition of physioxia is 5% CO₂ and 3.0 to 7.4% O₂, and atmospheric condition of hypoxia is 5% CO₂ and 0.01 to 2.0% O₂. In preferred embodiments, the pre-cell model is incubated in hypoxia which is similar to in vivo tumor tissues to induce the lung cancer cell model (100) forms the VM vascular structure.

The fourth aspect of the present invention is to provide a method for establishing a lung cancer cell model (100) having vascular tubules, and the steps, cell types, materials, culturing conditions and other parameters thereof are overall the same as those in any one of the first aspect, the second aspect or the third aspect of the present invention, but the lung cancer cells (la) comprise first lung cancer cells expressing wild-type EGFR at a first basal level, or second lung cancer cells expressing mutant EGFR at a second basal level.

Similar to that in the first aspect of the present invention, the first lung cancer cells or the second lung cancer cells are derived from primitive cells of a lung cancer tissue or from a lung cancer cell line, wherein the lung cancer tissue can be retrieved from a cryo-preserved or a freshly removed lung cancer tumor, including small-cell lung cancer, large-cell lung cancer, squamous cell lung carcinoma or lung adenocarcinoma. The lung cancer cell line includes immortalized cell line derived from any one of the aforesaid lung cancers, such as A549, HCC827, H460, H1299, H1650 or H1975.

In some embodiments, mutant EGFR expressed in the second lung cancer cells can be a mutant EGFR with amino acid residue substitution. For instance, the second lung cancer cells derived from small-cell lung cancer may express EGFR (p.Pro733Leu), EGFR (p.Pro753Ser), EGFR (p.Gly719Ala), EGFR (p.Glu734Lys), EGFR (p. Val742Ala), EGFR (p.Glu746Lys), EGFR (p.Ser752Tyr), EGFR (p.Ser768Val), EGFR (p.Ser768Ile), EGFR (p.Ser768Ile), EGFR (p.His773Arg), EGFR (p.Val834Leu), EGFR (p.Leu858Arg), EGFR (p.Leu858Arg), EGFR (p.Leu858Gln) or EGFR (p.Gly735Ser). The second lung cancer cells derived from squamous cell lung carcinoma may express EGFR (p.Gly719Asp), EGFR (p.Leu861Gln), EGFR (p.Gly719Arg) or EGFR (p.Leu861Pro). The second lung cancer cells derived from lung adenocarcinoma may express EGFR (p.Gly719Asp), EGFR (p.Ala237Phe), EGFR (p.His773Val), EGFR (p.Leu833Phe), EGFR (p.Lys852Thr), EGFR (p.Leu861Gln) or EGFR (p.Gly719Cys).

In some embodiments, the lung cancer cells (la) are derived from a lung cancer cell line, and an expression vector having regulatory elements can be applied to transport a foreign gene encoding wild-type EGFR or mutant EGFR into the lung cancer cells (la) to induce overexpression thereof. The first lung cancer cells carry a first exogenous genetic material to express wild-type EGFR at a first expression level, wherein the first expression level is larger than the first basal level. The second lung cancer cells carry a second exogenous genetic material to express mutant EGFR at a second expression level, wherein the second expression level is larger than the second basal level. Conceivably, to distinguish the overexpressed wild-type EGFR and the overexpressed mutant EGFR, the expression vector can be designed to carry a proper tag sequence to express a protein tag such as HA tag, FLAG tag or HIS tag, or a fluorescent tag such as GFP, YFP or RFP.

In various embodiments, the first lung cancer cells further express LAMC2 at a third expression level, and the second lung cancer cells further express LAMC2 at a fourth expression level, wherein the fourth expression level is larger than the third expression level. Up-regulation of laminin subunit gamma-2 (LAMC2) in lung adenocarcinoma (LUAD) has been considered relevant to tumor invasion, potential of metastasis and malignant prognosis. Being a member of ECM proteins, LAMC2 plays a significant role in basement membrane. LAMC2 is also involved in regulatory mechanisms of EGFR protein expression, and changes in its expression level may indirectly regulate 3D-VM formation and cell invasion of EGFRmt LUAD.

The fifth aspect of the present invention is to provide a platform for lung cancer medicine screening comprising a lung cancer cell model prepared by a method according to any one of the first aspect, the second aspect, the third aspect or the fourth aspect of the present invention.

The sixth aspect of the present invention is to provide a method for screening lung cancer medicines. With reference to FIG. 3, the method comprises:
cell model establishing step (A1): providing a lung cancer cell model (100) to be tested having vasculature of a first vascular density, wherein the lung cancer cell model to be tested is prepared by a method according to any one of the first aspect, the second aspect, the third aspect or the fourth aspect of the present invention;
medicine co-culturing step (A2): co-culturing the lung cancer cell model (100) to be tested and an anti-cancer medicine for a screening time so as to obtain a tested lung cancer cell model having vasculature of a second vascular density; and
therapeutic effectiveness evaluating step (A3): evaluating quantitative relationship between the first vascular density and the second vascular density to determine whether the anti-cancer medicine is therapeutically effective, wherein:
when a ratio of the first vascular density to the second vascular density is larger than 1, determining the anti-cancer medicine to be therapeutically effective; when a ratio of the first vascular density to the second vascular density is equal to or smaller than 1, determining the anti-cancer medicine to be therapeutically ineffective.

In various embodiments, the first vascular density and the second vascular density refer to a tubular density index obtained according to image analysis of 3D-VM vasculature. The tubular density index includes lumen area, total tube length or total tube area. To compare vascular density changes before and after medicine treatment, the first vascular density refers to a first lumen area, a first total tube length or a first total tube area, and the second vascular density refers to a second lumen area, a second total tube length or a second total tube area.

Particularly, the quantification of lumen area of 3D-VM tubular structure is based on one single frame real-time image. At least 3 or more area of interest (AOI) are involved in mean ROI (region of interest) calculation. Each ROI represents a selected vascular tube or vascular lumen within each tested cell model. Following AOI selection, the mean ROI was calculated by aggregating the ROIs for a plurality of AOIs and dividing this sum, thereby deriving an average area that reflects the typical dimensions of the regions analyzed in each individual cell model. On the other hand, the standard average was ascertained by dividing the mean ROI by the mean total tube average. This ratio provides a standardized metric, enabling the effective comparison of anti-3D VM efficiency across varying experimental setups or among different cell lines, and more precisely demonstrate disruptive effect of the anti-cancer medicine against 3D-VM. In some exemplary embodiments, the entire quantification process can be facilitated by employing the Angiogenesis Analyzer for ImageJ.

In various embodiments, the anti-cancer medicine to be tested can be EGFR tyrosine kinase inhibitors (TKIs), cell cycle blockers, or apoptosis-promoting reagents. Exemplarily, the anti-cancer medicine can be Foslinanib, Osimertinib, Palbociclib, Dinaciclib, Erlotinib, Ibrutinib, Imatinib, Venetoclax, Vorinostat, Bortezomib, Sorafenib, Gemcitabine or any combination of two or more thereof. Preferably, the anti-cancer medicine comprises Foslinanib, Osimertinib, or a combination thereof.

### 1. Materials and Methods

### 1.1 Cell Culture and Reagents

The cell lines A549, H1299, B16F10, HCC827, and H1650 were procured from the American Type Culture Collection (ATCC). The H1299-EGFR-L858R mutant cell line was generously provided by Professor Jan-Jong Hung from the Department of Biotechnology and Bioindustry Sciences at National Cheng Kung University, Tainan, Taiwan. The maintenance of the H1299, H1299-EGFR-wild type, H1299-EGFR-L858R, HCC827, and H1650 cell lines was carried out in RPMI-1640 medium, whereas the B16F10 cell line was cultured in Dulbecco's Modified Eagle Medium (DMEM), and the A549 cell line in F12K medium. All media were supplemented with 10% fetal bovine serum (FBS, Gibco) and 1% antibiotic-antimycotic solution (Gibco). Cultivation of all cell lines was performed in a controlled environment at 37° C. with a 5% CO₂ atmosphere.

### 1.2 Matrigel Tube Formation Assay

The assessment of in vitro vasculogenic mimicry (VM) capabilities of cancer cells was conducted utilizing the Matrigel tube formation assay. Chamber slides (TruLive3D Dishes, Burker) were prepared by applying a 100 μl layer of growth factor-reduced Matrigel (Corning) and subsequently allowing for gel solidification over a 30-minute period at 37° C. Following solidification, a cell suspension prepared in culture media at a concentration of 1.25×10⁵ cells per 150 μl was evenly distributed over the gel surface. This preparation was then incubated for an additional 30 minutes at 37° C. within a CO₂ incubator. After this initial incubation period, an overlay of 1× Matrigel mixed with culture medium was applied directly to the cells, so as to obtain a pre-cell model (10), and followed by further incubation at 37° C. for 5 to 24 hours so that a lung cancer cell model (100) was obtained for subsequent 3D-VM observation.

### 1.3 Three-Dimensional (3D) Hypoxic-VM Dynamics

Initially, the cell lines to be tested were seeded in chamber slides following the procedures in the aforesaid section 1.2 so as to obtain the lung cancer cell model (100). Subsequently, the chamber slides were placed alongside a 10 cm dish filled with sterilized MQ (Milli-Q), ensuring adequate humidity levels were maintained within the hypoxia chamber. Hypoxia was induced by aerating the chamber with a gas mixture composed of 5% CO₂, 1% 02, and 94% N₂ for a duration of 5 minutes to reach equilibrium. Following this aeration phase, the valve was sealed to arrest the gas flow, thereby preserving the hypoxic environment. After the chamber encapsulating the chamber slides in induced hypoxia, it was subsequently transferred to an incubator maintained at 37° C. The incubation period was tailored to the specific objectives of each experiment, facilitating detailed observation and analysis of VM structure development, including tubes and lumens, under meticulously controlled hypoxic conditions. Observations of the 3D-VM vasculature were conducted at intervals of 24, 48, and 72 hours post-induction using light sheet microscopy. The collected data were then analyzed utilizing the Imaris system for comprehensive insights into the morphological adaptations of the cancer cells and 3D-VM vasculature in response to hypoxic stress.

1.4 3D Co-Culture with Microspheres

First, a 10 μL aliquot of 10× concentrated Matrigel was evenly distributed across the base of each culture tray. These trays were then incubated at 37° C. for at least 10 minutes, allowing the Matrigel to solidify and form a uniform gel layer, which serves as a scaffold for cell attachment and growth. Following the preparation of the Matrigel layer, a cell suspension comprising 4×10⁴ cells mixed with Carboxylate-modified microsphere (F8821, Invitrogen; 1 μm) or Amine-modified purchased (F8763, Invitrogen; 0.2 μm) at a dilution ratio of 1:1000 in 15 μL of medium was prepared. This cell suspension was then meticulously layered over the previously solidified Matrigel in each tray. To ensure optimal cell adhesion to the Matrigel surface, the trays were placed back into the incubator at 37° C. for an additional 15 minutes. After the seeding of cells, the nuclei were stained to facilitate subsequent visualization and analysis. This was achieved by diluting a nucleus-specific dye with 1× Matrigel to attain the appropriate staining concentration, which was then applied to the co-culture system. The trays were incubated for 24 hours to allow for comprehensive integration of the staining, facilitating detailed examination of cell-microsphere interactions within this engineered 3D microenvironment.

### 1.5 3D Culture PAS Staining

The lung cancer cell model (100) was fixed in 4% formaldehyde for 20 minutes and washed with PBS and 1×TBS for 15 minutes, respectively. Next, the lung cancer cell model (100) was incubated with 0.5% periodic acid for 3 minutes, washed with distilled water three times, and treated with Schiff buffer for 10 minutes. After washing with distilled water, the lung cancer cells (1a) were stained with Hematoxylin for 5 minutes and then passed again. Finally, the stained lung cancer cell model (100) was observed under the light microscope (Olympus BX50).

### 1.6 CCLE Transcriptomics Analysis

The vasculogenic mimicry (VM) metagene signature was obtained from the report by Velez DO et al. (Nat Commun 8, 1651, 2017). The gene expression data of melanoma and lung cancer cells from the Cancer Cell Line Encyclopedia (CCLE) were downloaded from https://data.broadinstitute.org/ccle/CCLE_DepMap_18Q2_RNAseq_RPKM_20180502.gct. The demographic data and mutational status of the cell lines were obtained from cBioPortal (https://www.cbioportal.org/). The VM genes' activity score for each sample was calculated using the Single-Sample Gene Set Enrichment Analysis (ssGSEA) module from GenePattern, and the ssGSEA scores were Z-transformed. The boxplot and group mean comparison were calculated using the ggpubr package in R. Bioinformatic analysis was performed in R programming language (version 4.2.1).

### 1.7 EGFR Wild-Type Plasmid Transfection

H1299 cells were cultured until reaching 70 to 80% confluency at 37° C. with 5% CO₂ to ensure optimal growth and viability. The EGFR wild-type plasmid DNA, cloning vector: pcDNA3.1 (−) or pEGFP-N1+, was prepared in a serum-free growth medium, and Lipofectamine 3000, or an equivalent transfection agent, was used to facilitate the gene's entry into the cells. The diluted EGFR wild-type plasmid was mixed with reagents and incubated for 15 to 20 minutes at room temperature before being added to the H1299 cells and incubated at 37° C. in a CO₂ incubator. After 24 hours of stable transfection, G418 (500 μg/mL) was introduced into the medium, with the G418-containing medium refreshed every 2 to 3 days based on the cell growth rate. Following transfection, cells were maintained in a fresh, complete growth medium, with subsequent monitoring for EGFR expression. The effectiveness of the transfection and the impact of EGFR expression were evaluated using various analytical methods such as Western blotting, qRT-PCR, and immunofluorescence staining. Additionally, changes in cellular behavior, including proliferation, migration, and invasion, were assessed.

1.8 Fluorescence-Activated Cell Sorting (FACS) Method for Isolating pEGFP-N1+Cells

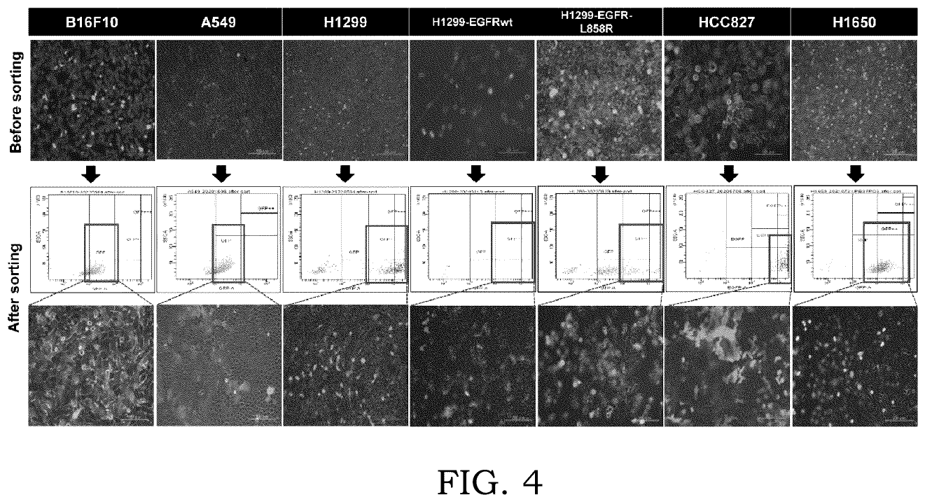

Cells after transfection were subjected to trypsinization, subsequently washed thrice with phosphate-buffered saline (PBS), and resuspended in PBS to maintain viability and prevent clumping. The population of cells expressing enhanced green fluorescent protein (pEGFP-N1+) was then identified and isolated using a FACS system. Please refer to FIG. 4, morphologies prior- and post-sorting of stable cell lines including B16F10, A549, H1299, H1299-EGFR-wild type, H1299-EGFR-L858R, HCC827, H1650 were present under fluorescent microscopy. Post-sorting, the collected pEGFP-N1+ cells were preserved in suitable culture media for subsequent expansion and experimentation.

### 1.9 Cell Proliferation Assay

Cell viability was determined using an MTT assay. Cells were plated at a density of 4×10³ cells/well within 96-well plates. These cells were then exposed to various concentrations of anti-vasculogenic mimicry (anti-VM) compounds across different time points: 0, 24, 48, and 72 hours. Following the treatment period, the MTT reagent (sourced from Sigma) was added to each well, and the cells were incubated at 37° C. for 2 hours. Subsequently, the plates were centrifuged at 1500 rpm for 10 minutes to facilitate the removal of the supernatant. The resultant formazan crystals were dissolved using dimethyl sulfoxide (DMSO), and the absorbance of the solution, indicative of cell viability, was measured at a wavelength of 492 nm using an ELISA plate reader.

### 1.10 Two-Dimensional (2D) VM Networks Inhibition Assay Via High-Content Imaging

Cells to be tested were plated in 96-well plates (manufactured by Greiner) and allowed to adhere overnight under standard culture conditions. Following attachment, the cells to be tested were exposed to various concentrations of anti-VM compound foslinanib (ABEx BIO-Research Center). The treatment's efficacy, specifically its ability to inhibit VM vasculature, was monitored time-dependent. The assessment of 2D-VM network disruption was conducted using the ImageXpressR Micro Confocal High-Content Imaging System (Molecular Devices LLC). This advanced imaging technology facilitates high-resolution visualization of cell morphology and tubular networks. Analysis of the imaging data was performed using MetaXpress software, allowing for quantitative measurement of 2D-VM network inhibition. This methodological approach provides a robust framework for evaluating the potential of anti-VM compounds.

### 1.11 Quantification of Three-Dimensional (3D) VM Lumens

The quantification of tube or lumen formation of 3D-VM vasculature was conducted utilizing an analytical approach on at least eight distinct areas of interest (AOIs), among which three AOIs were specified as representative sections of images. The region of interest (ROI) indicates a delineated patent tube or hollow lumen within each tested cell. First, the AOI was identified and quantified. Following this, the mean ROI was calculated by aggregating the ROIs for all eight AOIs (area 1+ area 2+ . . . area 8) and dividing this sum by eight, thereby deriving an average area that reflects the typical dimensions of the regions analyzed in each individual lung cancer cell model. Finally, the standard average was ascertained by dividing the mean ROI by the mean total tube average. This ratio provides a standardized metric, enabling the effective comparison of anti-3D-VM vasculature efficiency across varying experimental setups or among different cell lines; the formula is as follows:

$$Tube or lumen area degradation score : \frac{MEAN ROI}{MEAN TOTAL TUBE AREA} \div Standard average of mock$$

The entire quantification process was facilitated by employing the Angiogenesis Analyzer for ImageJ.

### 1.12 Protein Sample Preparation and Western Blot Analysis

Cells were seeded at a density of 2×10⁶ cells in a 6 cm culture dish for protein sample preparation. The cultured cells were washed twice with ice-cold 1× phosphate-buffered saline (PBS) and then lysed with RIPA buffer containing a 25× protease inhibitor cocktail (Roche) on ice. After centrifugation, the cell lysate samples were boiled with 6X sample buffer for 10 minutes. The cell lysate protein samples were separated by SDS-PAGE and transferred to polyvinylidene difluoride (PVDF) membranes (Millipore). The membranes were then blocked with 5% skim milk (HIMEDIA) dissolved in Tris-buffered saline Tween (TBST) buffer for 1 hour, followed by three 10-minute washes with TBST. The membranes were incubated with primary antibodies, including LAMC2 (ab210959), EGFR (D38B1) (#4267), Phospho-EGFR (Tyr992) (4478G), and Actin (MAB1501), overnight at 4° C. Subsequently, the membranes were incubated with the appropriate secondary antibodies for 1 hour at room temperature. Protein expressions were visualized using Western Lightning Plus ECL (PerkinElmer), detected by the iBright FL1000 imaging system, and analyzed using ImageJ software.

### 1.13 PAS/CD31 Dual Staining and Immunohistochemistry (IHC) Assay

The formalin-fixed, paraffin-embedded tissue sections were deparaffinized in xylene and rehydrated through a graded ethanol series. Antigen retrieval was performed using a citrate buffer (pH 6.0) under high pressure for 5 minutes. Endogenous peroxidase activity was quenched with 3% hydrogen peroxide for 10 minutes. Sections were then blocked with 5% bovine serum albumin (BSA) for 1 hour at room temperature. After washing, the sections were incubated with horseradish peroxidase (HRP)-conjugated secondary antibody, followed by diaminobenzidine (DAB) substrate. Subsequently, sections were subjected to Periodic Acid-Schiff (PAS) staining, which targets polysaccharides and mucosubstances in the extracellular matrix and basement membrane, producing a magenta color. Primary antibody specific to proteins associated with VM, laminin-5y2 (LAMC2) was applied to the sections and incubated overnight at 4° C. Finally, the sections were counterstained with hematoxylin and mounted for microscopic examination. Human tissue samples consisted of lung adenocarcinoma specimens, categorized into two groups based on EGFR status: wild-type EGFR and EGFR mutations. All diagnoses were confirmed by histopathology. The collection of all tissue samples was approved by the institutional review board of the National Cheng Kung University Hospital (IRB no: B-ER-108-090).

### 2. Results

### Example 1: Establish a Novel Tool to Investigate 3D VM Dynamics

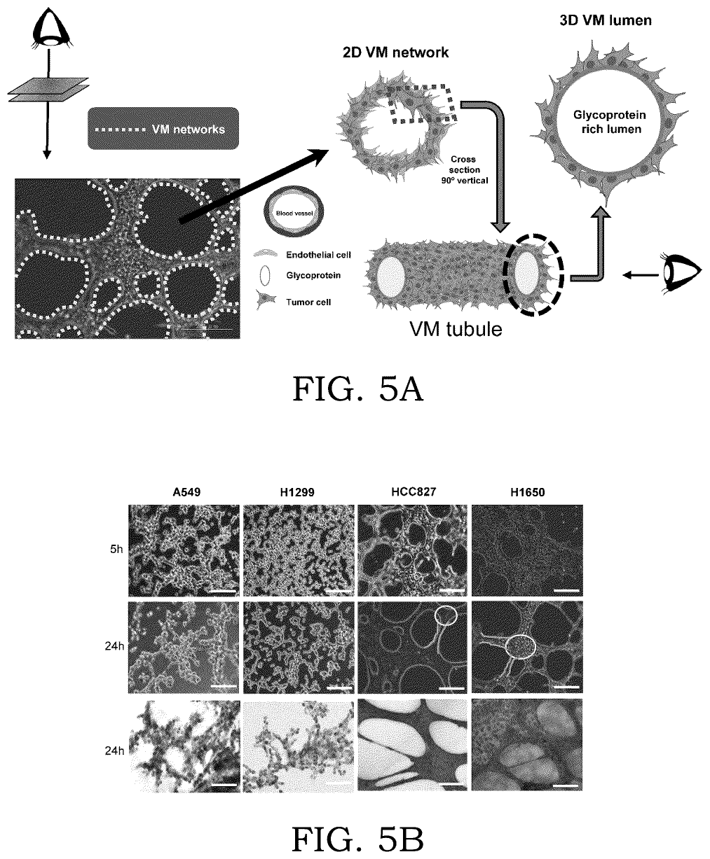

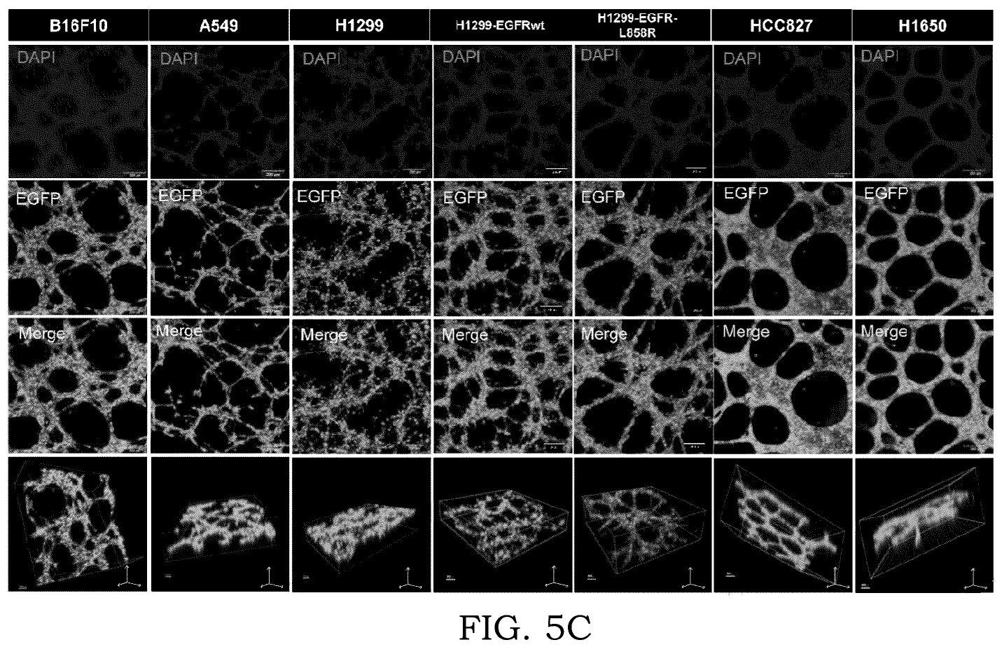

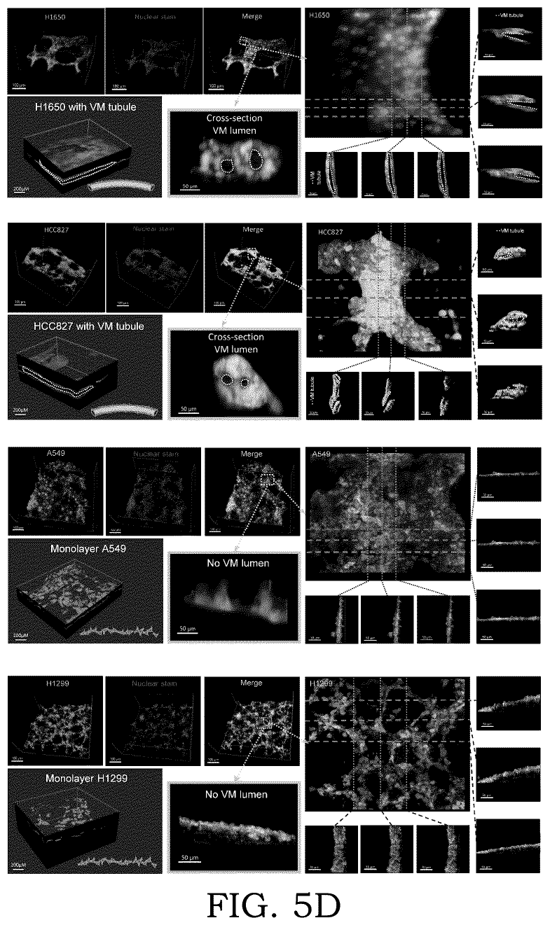

A common pitfall in vasculogenic mimicry is misclassifying 2D-VM networks as 3D-VM vasculature (FIG. 5A). The MDA-MB-231 and BxPC-3 cancer cell lines have been demonstrated to be able to form VM networks characterized by numerous vertices and edges. Being observed from the top view (along z-axis to observe the x-y plane), as presented in FIG. 5A (left), vertices represent nodes, while edges indicate connections between nodes (yellow dotted line).

As presented in FIG. 5B, the top view of EGFR mutated cells H1650 and HCC827 revealed interconnected rigid edges (labeled with magenta line) forming vertices (labeled with yellow circle) in contrast to EGFR wild-type cells A549 and H1299.

To distinguish 2D-VM networks from 3D-VM vasculature, in vitro dynamic 3D-VM vasculature model was developed by using the murine melanoma cell line B16F10. Fluorescently labeled cells were utilized for real-time observation.

To ensure that the 2D-VM network truly corresponds to the tube and lumen structures of 3D-VM vasculature rather than a monolayer cell structure, fluorescently labeled cell lines were utilized to establish cell models having dynamic 3D-VM vasculature. As presented in FIG. 5C, through microscopic examination using FV3000 confocal microscope (FV3000) and image analysis using Imaris software, cell models built with stable cell lines fluorescence-enriched B16F10, A549, H1299, H1299-EGFR-wild type, H1299-EGFR-L858R, HCC827, H1650 after FACS screening was presented, and the scale bar represents 100 μm. Real-time visualization of 3D-VM tubular formation was demonstrated.

Please refer to FIG. 5D, four EGF-transfected non-small cell lung cancer cell lines (H1650, HCC827, A549, H1299) were used to establish lung cancer cell models having 3D-VM vasculature, revealing variations in the network topology across these cell lines. Notably, H1650 and HCC827 cells formed network-like patterns with vertices and edges, whereas A549 and H1299 cells aggregated and clustered. To confirm the presence of patent tubes and hollow lumens in lung cancer cells, after a 24-hour Matrigel-based culture, average lumen diameters ranging between 25 to 50 μm in the H1650 and HCC827 cell models were observed. In contrast, A549 and H1299 cells exhibited monolayer characteristics without patent tubes or hollow lumens. The multi-region longitudinal tubes and cross-section lumens were captured through microscopic examination using light sheet fluorescence microscopy (LSFM) and image analysis using interactive microscopy image analysis software (IMARIS) for z-stack reconstruction.

### Example 2: Hypoxic VM Dynamics Model

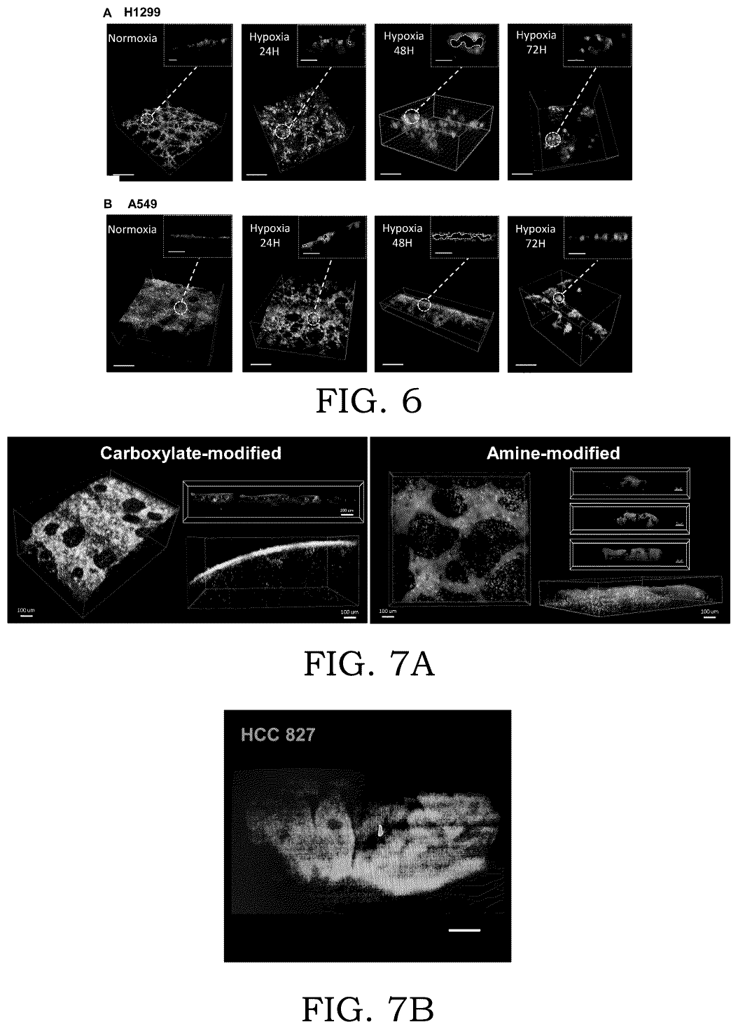

To elucidate the potential of lung cancer cells, initially devoid of 3D-VM vasculature under normoxic conditions, a 3D model simulating hypoxic 3D-VM structure was conducted in Example 2 to develop such features under hypoxic stress. As shown in FIG. 6, a significant induction of 3D-VM vasculature in H1299 cells could be observed after 48 hours of exposure to hypoxia. Notably, by the 72-hour mark, a pronounced degradation of the 3D-VM vasculature was observed, indicating a transient nature of 3D-VM vasculature under sustained hypoxic conditions.

Similarly, A549 cells began to exhibit 3D-VM vasculature as early as 24 hours following hypoxia induction, with an apparent increase in the complexity and number of tubular structures by the 48-hour time point. However, as shown in FIG. 6, similar to the H1299 cells, the 3D-VM vasculature of A549 cells also showed signs of deterioration by 72 hours.

Example 2 not only underscores the capacity of lung cancer cell models to adapt to hypoxic stress and obtain endothelial characteristics to form 3D-VM vasculature, but also highlights the transient stability of the 3D-VM vasculature over time. The observed dynamics offer crucial insights into the cellular behaviors and mechanisms underpinning transient VM formation during tumor cell metastasis.

### Example 3: Lumen Patency of 3D-VM Vasculature

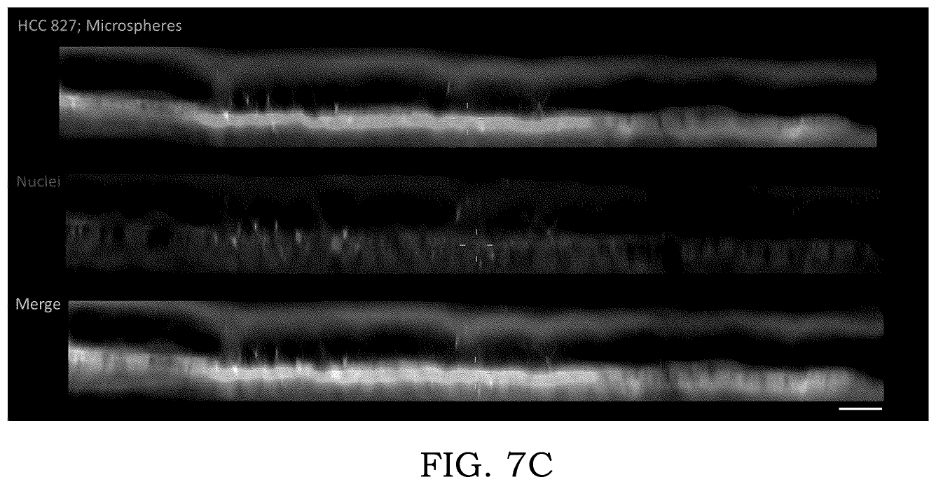

In Example 3, an innovative exploration to ascertain the functionality of 3D VM structures was conducted by implementing a 3D co-culture system of lung cancer cells enriched with microspheres. As shown in FIG. 7A (left), the co-culture of HCC827 lung cancer cells with carboxylate-modified microspheres yielded fascinating results, demonstrating significant integration of the microspheres within the VM tubules and lumens fabricated by the HCC827 cells and indicating that 3D-VM vasculature fabricated by HCC827 cells not only provides patent tubes and lumens, but also hints at their potential functionality as conduits or supportive structures within a tumor microenvironment. In another aspect, as shown in FIG. 7A (right), the co-culture involving amine-modified microspheres presented a markedly different interaction pattern. These microspheres predominantly adhered to the external cell membranes of HCC827 cells and were largely found within the confines of the Matrigel substrate, showing minimal incorporation into the 3D-VM vasculature. Example 3 demonstrates that surface modification of the microspheres significantly influences the interaction between the microspheres and 3D-VM vasculature, which could be a reference marker for the evaluation of tubular patency of 3D-VM vasculature.

In light of this, please refer to FIG. 7B presenting a partial magnified x-z cross-section VM tube within an HCC827 cell model, carboxylate-modified microspheres were observed in the tubular section. With reference to FIG. 7C presenting a motion picture screenshot of y-z cross-section VM tube within a HCC827 cell model, multiple carboxylate-modified microspheres were observed in a lumen of the longitudinal tube. The VM tube was verified to be a patent tube with functionality. Formation of 3D-VM vasculature was ascertained in the HCC827 cell model.

### Example 4: Foslinanib Disrupts 3D-VM Structure

To evaluate the reliability of a lung cancer cell model having dynamic 3D-VM vasculature serving as anti-vasculogenic mimicry (VM) drug screening platform, in Example 4, Foslinanib was applied as a test compound. The chemical structure of foslinanib, a phenyl-quinoline derivative, exhibits strong anti-neoplastic and antimutagenic properties.

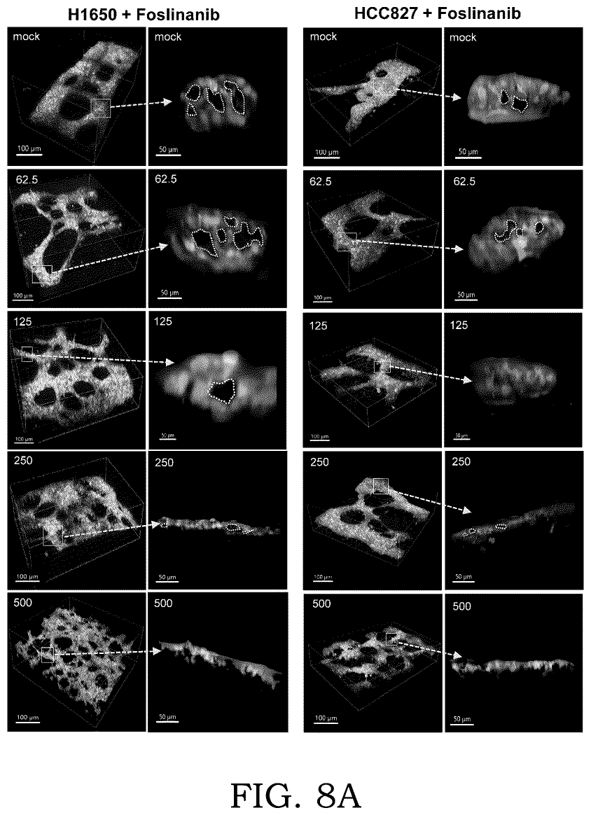

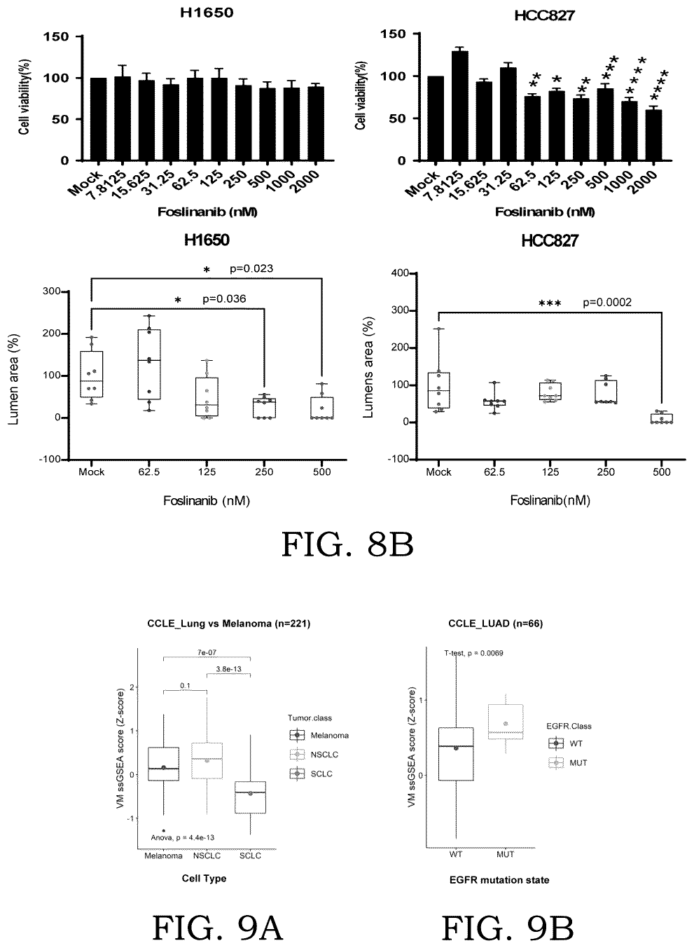

H1650 and HCC827 were used as VM-positive cell lines for anti-3D-VM vasculature testing. As shown in FIGS. 8A to 8b, real-time LSFM observation revealed changes in 3D-VM vasculature and cell viability of H1650 and HCC827 lung cancer cell models under various foslinanib concentrations. In H1650 lung cancer cell model, foslinanib presents no significant cell toxicity against H1650 cells but presents disruption of VM lumens at 62.5 nM and significant inhibition of 3D-VM vasculature at 250 nM and 500 nM. As shown in FIG. 8B (bottom), lumen area was significantly reduced at 250 nM and 500 nM. On the other hand, in HCC827 lung cancer cell model, foslinanib demonstrated apparent cell toxicity against HCC827 cells at 62.5 nM, and cell viability decreased to 70%. Moreover, the lumen area showed significant reduction after 24-hour incubation at 500 nM, exhibiting a similar inhibitory effect on 3D-VM vasculature.

In light of the foregoing, Example 4 validated the applicability of the lung cancer cell model having 3D-VM vasculature to anti-VM drug screening and evaluation of VM drugs' inhibitory and disrupting effects on the development of 3D-VM vasculature.

### Example 5: Interplay of VM and EGFR Mutation

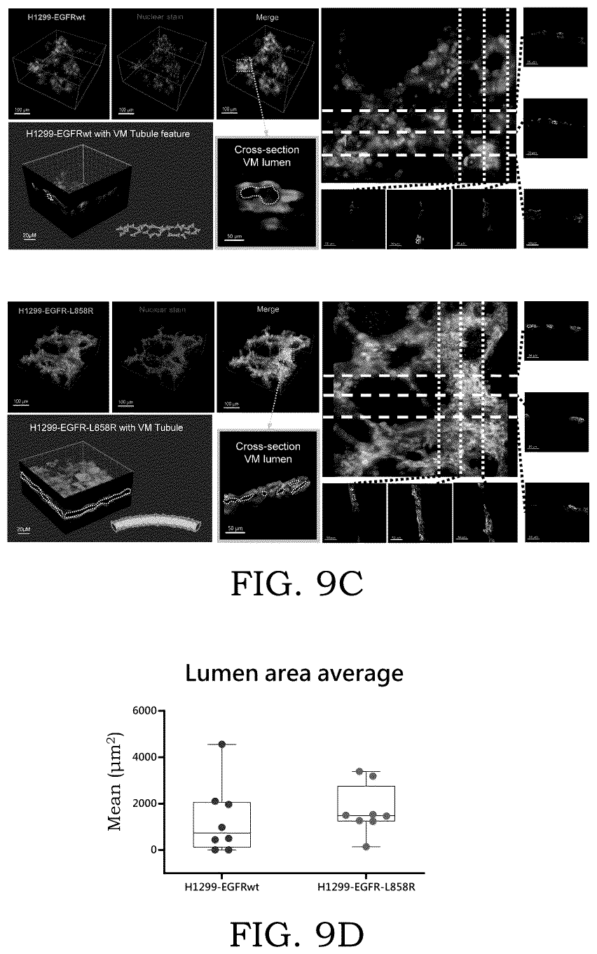

In Example 5, a bioinformatic analysis was conducted on lung cancer datasets, including non-small cell lung cancer (NSCLC) and small cell lung cancer (SCLC) from the CCLE public database. As shown in FIG. 9A, the melanoma dataset served as a reference for relative VM positivity. The VM ssGSEA scores of the lung adenocarcinoma (LUAD) dataset from the CCLE public database were analyzed. The samples were divided into groups based on EGFR mutation status and EGFR wild type. The VM ssGSEA score was notably higher in samples with EGFR mutations (FIG. 9B), suggesting a higher likelihood of VM occurrence in LUAD with EGFR mutations compared to EGFR wild-type cases.

With reference to FIG. 9C, H1299-EGFRwt cells exhibited a distinct tubularization pattern, while H1299-EGFRmt cells demonstrated significant 3D-VM patent tubes and hollow lumens. Notably, the intercellular tubules of H1299-EGFRwt cells displayed a less cohesive structure with sporadic lumens when compared to H1299-EGFRmt cells. As shown in FIG. 9D, with quantitative analysis, the average lumen area of H1299-EGFRmt cells is significantly larger than that of H1299-EGFRwt, which implicates that overexpression of EGFRmt promotes H1299 cells to form 3D-VM vasculature having lumens with functionality.

Moreover, as shown in FIG. 5D, in 2D-VM network fabricated by H1299 lung cancer cell line, there was a lack of observable lumens in the 3D model experiment and a lack of tubes with patency. Compared with this, stable cell line H1299-EGFRmt demonstrated hollow lumens and tubes with patency, as shown in FIG. 9C (bottom).

### Example 6: Efficacy of Combine Anti-VM and EGFR TKI

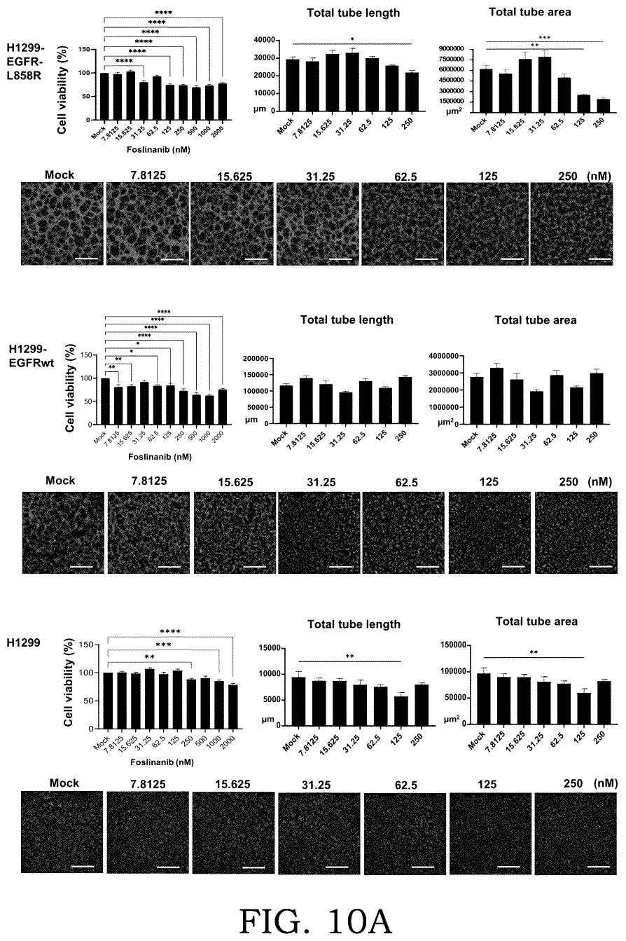

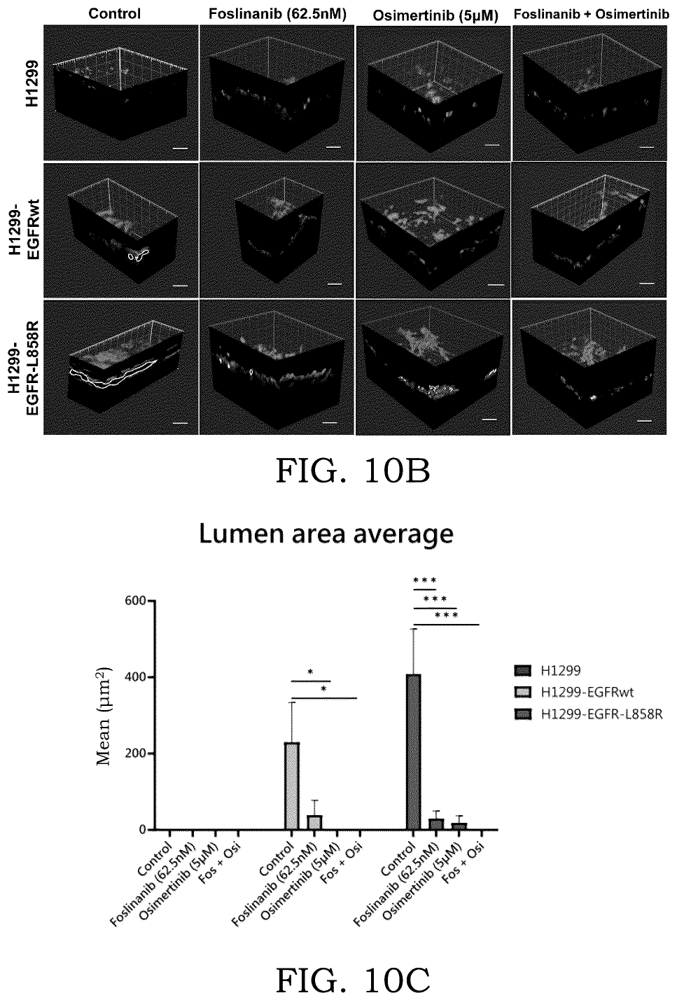

FIG. 10A illustrated cell viability and 2D-VM network dynamics of H1299-EGFRmt cell model, H1299-EGFRwt cell model and H1299 cell model. Within the range of 7.8125 nM to 500 nM, H1299-EGFRmt cell model showed a significant decrease in cell viability at 31.25 nM, and 2D total network length and total network area showed a decreasing trend at any concentration beyond 31.25 nM. Compared with H1299-EGFRwt cell model, the inhibitory effect of foslinanib on the formation of a 2D-VM network at high concentration (beyond 31.25 nM) was rather insignificant, demonstrating a worse therapeutic effect. In another aspect, cell viability of H1299 cell model was suppressed at a high concentration of foslinanib (beyond 250 nM). Because no 3D-VM vasculature was observed in the H1299 cell model using 3D-VM methodology, Example 6 focused on 2D-VM network inhibitory effect of foslinanib. The 2D total network length and total network area of the H1299 cell model showed less effective inhibition when compared with that of the H1299-EGFRmt cell model.

Example 6 further combined EGFR tyrosine kinase inhibitors (TKIs) with anti-angiogenic agents to overcome resistance mechanisms and enhance treatment responses by targeting multiple pathways simultaneously. Although the third-generation EGFR TKI, osimertinib, showed no additional benefit when combined with anti-angiogenic therapy. To investigate the influence of 3D-VM vasculature of a combination treatment of EGFR TKI osimertinib and the anti-VM agent foslinanib, Example 6 employed H1299 cells transfected with EGFR wild-type (H1299-EGFRwt) and EGFR-L858R(H1299-EGFRmt) as testing cell lines.

FIG. 10B showed that the control group H1299 lung cancer cells did not develop 3D-VM vasculature. In contrast, the H1299-EGFRwt cell model presents a distinct tubular pattern characterized by loosely connected tubes and sporadic lumens, while the H1299-EGFRmt cell model formed significant 3D-VM vasculature with patency.

With reference to FIGS. 10B to 10C, foslinanib demonstrated disruptive effect on 3D-VM vasculature of H1299-EGFRwt cell model at 62.5 nM. Although, overexpression of EGFRmt promoted H1299 cells to form 3D-VM vasculature, quantitative analysis showed that disruption of 3D-VM vasculature in a H1299-EGFRmt cell model by foslinanib was significant. Compared with it, osimertinib at an extremely high concentration of 5/M did not impact the formation of 3D-VM vasculature. Still, such high concentration did demonstrate cytotoxic effects on the H1299 cell model, H1299 EGFRwt cell model, and H1299mt cell model. Therefore, the quantitative analysis also showed that 3D-VM vasculature was significantly disrupted after osimertinib treatment. Furthermore, combining osimertinib at 5 μM and foslinanib at 62.5 nM disrupted 3D-VM vasculature from perspectives of 3D imaging and quantitative analysis. The 3D-VM vasculatures in both H1299-EGFRwt cell model and H1299-EGFRmt cell model were almost completely inhibited.

### Example 7: Overexpression of Laminin Subunit Gamma-2 in 3D-VM Vasculature in EGFRmt Lung Cancer Cell Model

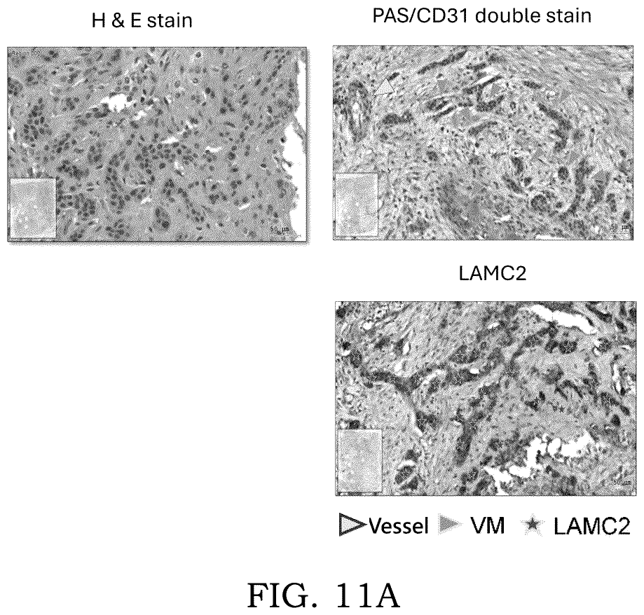

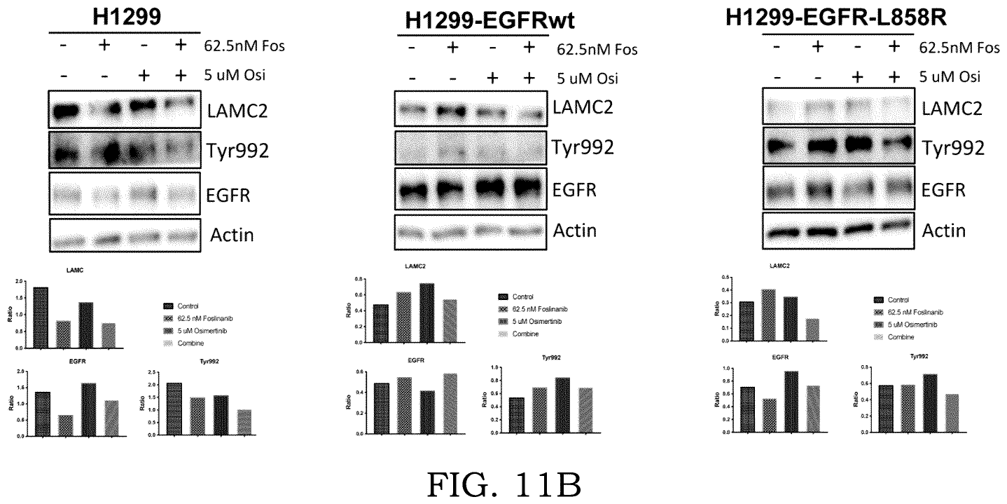

To evaluate tumor vasculature, immunohistochemistry was conducted on LUAD sample tissues. As shown in FIG. 11A, blood vessels and regions resembling VM were identified by using PAS-CD31 double staining on formalin-fixed paraffin-embedded (FFPE) tissues. On the one hand, the PAS stain highlighted the VM area with a magenta cord-like signal (indicated by orange arrowheads). On the other hand, VM area with PAS-CD31 double staining was also highlighted with LAMC2 via immunohistochemistry (denoted by red stars), showing that LUAD tumors with EGFR mutation exhibited significantly more VM structures and higher LAMC2 expression compared to their wild-type counterparts. In light of immunochemistry results, 3D-VM vasculature formation in LUAD tumors may involve LAMC2.

The total expression levels of phosphorylated and total EGFR, LAMC2 were quantified using Western blot analysis in H1299 cell model, H1299-EGFRwt cell model, and H1299-EGFRmt cell model As indicated in FIG. 11B, the data revealed that osimertinib markedly reduced the levels of phosphorylated EGFR. Treatment with foslinanib resulted in a significant decrease in LAMC2 protein levels across the examined cell lines. Moreover, concurrent administration of osimertinib and foslinanib led to a synergistic reduction in the expression of both phosphorylated EGFR and LAMC2, indicating a potential interplay between the EGFR-L858R mutation and vasculogenic mimicry mechanisms, implicating that EGFR-L858R can be a target candidate of anti-VM drugs.

1. A method for establishing a lung cancer cell model having vascular tubules comprising:
seeding a cell suspension between an upper 3D culture substrate and a lower 3D culture substrate to obtain a pre-cell model, wherein the cell suspension comprises lung cancer cells; and
incubating the pre-cell model for a cultivation time so as to obtain the lung cancer cell model, wherein:
both the upper 3D culture substrate and the lower 3D culture substrate are growth factor growth factor-reduced culture substrates; and
volume of the upper 3D culture substrate is larger than volume of the lower 3D culture substrate.

2. The method according to claim 1, wherein the pre-cell model is incubated in normoxia, physioxia or hypoxia.

3. The method according to claim 1, wherein the lung cancer cell comprises:
first lung cancer cells expressing wild-type EGFR at a first basal level, or the first lung cancer cells carrying a first exogenous genetic material to express wild-type EGFR at a first expression level, wherein the first expression level is larger than the first basal level; and
second lung cancer cells expressing mutant EGFR at a second basal level, or the second lung cancer cells carrying a second exogenous genetic material to express mutant EGFR at a second expression level, wherein the second expression level is larger than the second basal level.

4. The method according to claim 3, wherein the lung cancer cells are derived from a lung cancer tissue or a lung cancer cell line.

5. The method according to claim 1, wherein the pre-cell model obtaining step further comprises:
solidifying a lower volume of a lower 3D culture pre-substrate on a culture surface for a solidifying time so that the lower 3D culture substrate is formed;
incubating a middle volume of the cell suspension on the lower 3D culture substrate for an attachment time so that the lung cancer cells are attached to the lower 3D culture substrate; and
coating an upper volume of an upper 3D culture pre-substrate so that the upper 3D culture substrate is formed, thereby obtaining the pre-cell model, wherein the upper volume is larger than the sum of the lower volume and the middle volume.

6. The method according to claim 5, wherein:
the upper 3D culture pre-substrate comprises a first upper volume of upper cell basement membrane-like materials and a second upper volume of culture media, wherein the first upper volume is the same as the second upper volume, and the sum of the first upper volume and the second upper volume is equal to the upper volume; and
the lower 3D culture pre-substrate comprises lower cell basement membrane-like materials, the upper cell basement membrane-like materials are the same as or different from the lower cell basement membrane-like materials, wherein a ratio of the first upper volume, the second upper volume, the middle volume, and the lower volume is 1:(1.00 to 3.00):(1.05 to 1.65):(0.25 to 0.85).

7. The method according to claim 5, wherein the upper 3D culture pre-substrate is Matrigel and the lower 3D culture pre-substrate is Matrigel.

8. The method according to claim 5, wherein:
the cell suspension further comprises microspheres labeled with first fluorophores, and each of the microspheres comprises an electronegative organic group;
the lung cancer cells are further labeled with second fluorophores having emission light different from the emission light of the first fluorophores; and
the vascular tubule comprises a lumen surrounded by the lung cancer cells, and a tunica adventitia formed by the lung cancer cells surrounding on the lumen.

9. A platform for lung cancer medicine screening comprising a lung cancer cell model prepared by a method according to claim 1.

10. A method for screening lung cancer medicines, comprising:
providing a lung cancer cell model to be tested having vasculature of a first vascular density, wherein the lung cancer cell model to be tested is prepared by a method according to claim 1;
co-culturing the lung cancer cell model to be tested and an anti-cancer medicine for a screening time so as to obtain a tested lung cancer cell model having vasculature of a second vascular density; and
evaluating quantitative relationship between the first vascular density and the second vascular density to determine whether the anti-cancer medicine is therapeutically effective, wherein:
when a ratio of the first vascular density to the second vascular density is larger than 1, determining the anti-cancer medicine to be therapeutically effective; when a ratio of the first vascular density to the second vascular density is equal to or smaller than 1, determining the anti-cancer medicine to be therapeutically ineffective.

11. The method, according to claim 4, wherein the lung cancer cell line is selected from a group consisting of A549, HCC827, H460, H1299, H1650, and H1975.

12. The method according to claim 6, wherein the upper cell basement membrane-like materials comprise fibronectin, collagen, laminin, vitronectin, polylysine, sulfate proteoglycans, entactin or a combination thereof, and the lower cell basement membrane-like materials comprise fibronectin, collagen, laminin, vitronectin, polylysine, sulfate proteoglycans, entactin or a combination thereof.

13. The platform according to claim 9, wherein the pre-cell model is incubated in normoxia, physioxia or hypoxia.

14. The platform according to claim 9, wherein the lung cancer cell comprises:
first lung cancer cells expressing wild-type EGFR at a first basal level, or the first lung cancer cells carrying a first exogenous genetic material to express wild-type EGFR at a first expression level, wherein the first expression level is larger than the first basal level; and
second lung cancer cells expressing mutant EGFR at a second basal level, or the second lung cancer cells carrying a second exogenous genetic material to express mutant EGFR at a second expression level, wherein the second expression level is larger than the second basal level.

15. The platform according to claim 14, wherein the lung cancer cells are derived from a lung cancer tissue or a lung cancer cell line.

16. The platform according to claim 15, wherein the lung cancer cell line is selected from a group consisting of A549, HCC827, H460, H1299, H1650 and H1975.

17. The method according to claim 10, wherein the pre-cell model is incubated in normoxia, physioxia or hypoxia.

18. The method according to claim 10, wherein the lung cancer cell comprises:
first lung cancer cells expressing wild-type EGFR at a first basal level, or the first lung cancer cells carrying a first exogenous genetic material to express wild-type EGFR at a first expression level, wherein the first expression level is larger than the first basal level; and
second lung cancer cells expressing mutant EGFR at a second basal level, or the second lung cancer cells carrying a second exogenous genetic material to express mutant EGFR at a second expression level, wherein the second expression level is larger than the second basal level.

19. The method according to claim 18, wherein the lung cancer cells are derived from a lung cancer tissue or a lung cancer cell line.

20. The method according to claim 19, wherein the lung cancer cell line is selected from a group consisting of A549, HCC827, H460, H1299, H1650 and H1975.
